(19) United States
(12) Patent Application Publication
(10) Pub. No.: US 2013/0089581 A1
(43) Pub. Date: Apr. 11, 2013

(54) SKIN-FRIENDLY ADHESIVES FROM POLYALKLETHER-BASED PHOTOINITIATORS

(71) Applicants: Christian B. Nielsen, Copenhagen NV (DK); Niels Joergen Madsen, Alleroed (DK)

(72) Inventors: Christian B. Nielsen, Copenhagen NV (DK); Niels Joergen Madsen, Alleroed (DK)

(73) Assignee: Coloplast A/S, Humlebaek (DK)

(21) Appl. No.: 13/805,121

(22) PCT Filed: Jun. 22, 2011
(86) PCT No.: PCT/DK2011/050227

§ 371 Date: Dec. 18, 2012

(30) Foreign Application Priority Data
Jun. 22, 2010 (DK) PA201070282
Jun. 8, 2011 (DK) PA201170285

(51) Int. Cl. A61L 24/04

(52) U.S. Cl. CPC A61L 24/046
USPC 424/400; 522/170

(57) ABSTRACT

The invention provides a method for manufacturing a skin-friendly pressure-sensitive adhesive composition, said method comprising the steps of: a. providing a matrix composition comprising a polymeric photoinitiator of the general formula (I): R₁(A₁)ᵣ-(R₂(A₂)ₘ-0)ₒ-(R₃(A₃)n-O)ₚ—R4(A₄)ₛ (I) and b. curing the matrix composition in step a. by exposing it to UV radiation. The matrix composition may additionally comprise one or more adhesive-forming polymers and/or adhesive-forming monomers, or may simply consist of the polymeric photoinitiator of the general formula I, as defined herein. The invention also relates to the skin-friendly pressure-sensitive adhesive composition obtained by the method of the invention, as well as a medical device comprising said adhesive composition.

### FIELD OF THE INVENTION

The invention provides a method for manufacturing a skin-friendly pressure-sensitive adhesive composition. The invention also relates to the skin-friendly pressure-sensitive adhesive composition obtained by the method of the invention, as well as a medical device comprising said adhesive composition.

### BACKGROUND OF THE INVENTION

Pressure sensitive adhesives have for a long time been used for attaching medical devices, such as ostomy appliances, dressings (including wound dressings), wound drainage bandages, devices for collecting urine, orthoses and prostheses to the skin.

Due to the delicate nature of skin, there is a narrow window where a pressure-sensitive adhesive can function as a good and skin-friendly adhesive: on one hand, the adhesive should be able to attach the medical device to the skin and maintain it in place during use, while—on the other hand—removal of the medical device from the skin should not cause damage.

Examples of skin-friendly, pressure-sensitive adhesives are given in WO2009/006901 and WO2007/128320.

Curing of compositions through ultraviolet (UV) radiation requires efficient methods of initiating the chemical reaction responsible for the curing process. Cross-linking of polymeric material through generation of radical species upon irradiation with UV light is widely used to produce hydrogels. The photoinitiators used in UV curing processes can be either oligomeric or polymeric. Oligomeric photoinitiators and photoinitiators of low molecular weight are partially free to diffuse to the surface of the cured material (leach), thereby rendering these substances exposed to the environment. This poses particular problems in relation to medical devices, as the leached substances may then come into contact with the patient. Global regulation has developed to control the amount and nature of substances which may diffuse from a medical device designed to be in contact with a patient.

WO 2008/012325 and WO 2008/071796 describe photocuring of plastic coatings for medical devices, to provide such medical devices with lubricated surfaces.

Other published documents which relate to polymeric photoinitiators based on polyalkylethers are US 2007/0003588 and Xuesong Jiang et al, Polymer, 50 (2009) 37-41.

### OBJECT OF THE INVENTION

It is an object of the invention to provide a method for manufacturing a skin-friendly pressure sensitive adhesive composition, and the adhesive composition itself. In particular, it is an aim to reduce the problems associated with leaching/diffusion of substances from adhesive compositions and medical devices containing such adhesive compositions.

### SUMMARY OF THE INVENTION

It has been found by the present inventors that polymeric photoinitiators with certain structures can be used with benefits in the formation of adhesive compositions and medical devices.

The present invention therefore relates to a method for manufacturing a skin-friendly pressure-sensitive adhesive composition, said method comprising the steps of:
a. providing a matrix composition comprising a polymeric photoinitiator of the general formula I:

R₁(A₁)ᵣ-(R₂(A₂)ₘ-O)ₒ—(R₃(A₃)ₙ-O)ₚ—R₄(A₄)ₛ (I)
wherein R₂ and R₃ are independently at each occurrence identical or different, linear or branched alkylene or cycloalkylene groups; wherein R₂ and R₃ may be substituted with one or more substituents selected from CN; azides, esters; ethers; amides; halogen atoms; sulfones; sulfonic derivatives; NH₂ or Nalk₂, where alk is any C₁-C₈ straight chain alkyl group, C₃-C₈ branched or cyclic alkyl group;
R₁ and R₄ are independently at each occurrence identical or different, linear or branched alkyl or cycloalkyl groups or aryl groups or are independently at each occurrence selected from H, OH, CN, halogens, amines, amides, alcohols, ethers, thioethers, sulfones and derivatives thereof, sulfonic acid and derivatives thereof, sulfoxides and derivatives thereof, carbonates, isocyanates, nitrates, acrylates, polyethylenes, polyethylene oxides, polypropylene oxides, polyvinyl pyrrolidones, polypropylenes, polyesters, polyamides, polyacrylates, polystyrenes, and polyurethanes; and when R₁ and R₄ are alkyl and aryl groups, they may be substituted with one or more substituents selected from CN; OH; azides; esters; ethers; amides; halogen atoms; sulfones; sulfonic derivatives; NH₂ or Nalk₂, where alk is any C₁-C₈ straight chain alkyl group, C₃-C₈ branched or cyclic alkyl group;
o and p are each a real number from 0-5000 provided that o+p>0;
m and n are each a real number from 0-10,
provided that m+n>0;
r and s are each a real number from 0-5; and
A₁, A₂, A₃ and A₄ are identical or different photoinitiator moieties; and
b. curing the matrix composition in step a. by exposing it to UV radiation.

The matrix composition may additionally comprise one or more adhesive-forming polymers and/or adhesive-forming monomers, or may simply consist of the polymeric photoinitiator of the general formula I, as defined herein.

The invention also provides a skin-friendly pressure-sensitive adhesive composition obtainable via the method above, as well as a medical device comprising the adhesive composition.

### LEGENDS TO THE FIGURES

FIG. 1 illustrates a general motif of polymeric photoinitiators, with photoinitiator moieties pendant on a polyalkylether.

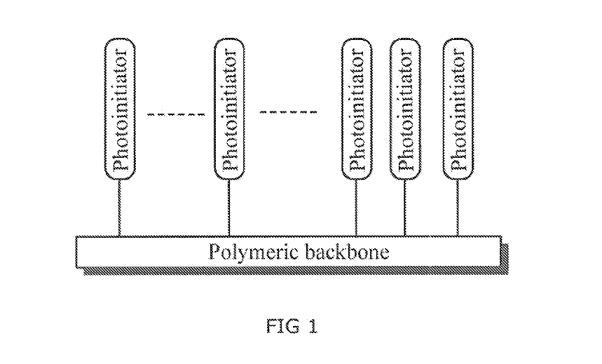

### DETAILED DISCLOSURE OF THE INVENTION

### Definitions

“Optionally-substituted” means optionally-substituted with one or more substituents selected from the group consisting of C₁-C₂₅ linear, branched or cyclic alkyl, aryl, —OH, —CN, halogens, amines, amides, alcohols, ethers, thioethers, sulfones and derivatives thereof, sulfonic acid and derivatives thereof, sulfoxides and derivatives thereof, carbonates, isocyanates, nitrates, acrylates. Preferably the one or more substituents are selected from the group consisting of —OH, —CN, halogens, amines, amides, alcohols, ethers, thioethers, sulfones and derivatives thereof, sulfonic acid and derivatives thereof, sulfoxides and derivatives thereof, carbonates, isocyanates, nitrates, acrylates. Most preferably, the substituent is selected from the group consisting of —OH, —CN, halogens, amines, amides, alcohols, ethers, thioethers, sulfones and derivatives thereof, sulfonic acid and derivatives thereof, and sulfoxides and derivatives thereof.

### Pressure-Sensitive Adhesive

Pressure-sensitive adhesives are those which form a bond when pressure is applied, and which do not require solvent, water, or heat to activate the adhesive. The bonding strength is dependent on the amount of pressure which is used to apply the adhesive to the surface. Several theories are used to describe the adhesive bonding and a few of these are exemplified in the following. Mechanical interlock theory accounts for simple filling of crevices, cracks and pores on the substrates which can be populated by the adhesive. Another theory is the adsorption theory, which accounts for adhesion by wetting: The adhesive wets the surfaces and then secondary intermolecular forces (van der Waal forces such as dipole-dipole, dipole-induced dipole and hydrogen bonds) accounts for the adhesive strength.

Some guidelines also exist for the rheological properties of pressure sensitive adhesives (see S. G. Chu, L-H. Lee (Eds.), “Dynamic mechanical properties of pressure sensitive adhesives”, Adhesive Bonding, Plenum Publishing, 1991, pp. 97-115.). When measuring both G′ and G″ at 0.1 Hz and at 100 Hz, the value of G′ should preferably be close to 10⁴ Pa at 0.1 Hz and 10⁵ Pa at 100 Hz, whereas G″ should be close to 10⁵ Pa at 0.1 Hz and close to 10⁴ Pa at 100 Hz. Molecular weight is related to the actual values of G′ and G″ inasmuch as the complex viscosity is related to with G′ and G″ as ω·η*=\sqrt{(G′)²+(G″)²}, where η* is the complex viscosity and ω is the angular frequency. In turn, the complex viscosity can be related to the real viscosity (η) through the Cox-Merz rule and further the viscosity is related to molecular weight through the Mark-Houwink relation (n=κ*M^{a}, where K and a are constants for a specific polymer type). From these relations it follows that an increasing value of the molecular weight is followed by an increase in \sqrt{(G′)²+(G″)²}. On the other hand an increase in either G′ or G″ will be manifested in a higher molecular weight. This serves to demonstrate, that if G′ and G″ are outside the window of values required for an adhesive it is possible to alter the molecular weight and possibly the degree of cross linking of the polymer such that optimal G′ and G″ values are obtained.

In order to test the adhesive properties themselves, several standards are available for evaluating the performance of pressure sensitive tapes; in particular ASTM D903 and ASTM D3330. In short, samples are prepared where the adhesive tapes are placed on rigid substrates (e.g. steel). The tapes are then peeled off at different angles using a tensile tester and the force needed to perform the peeling is monitored.

### Specific Embodiments of the Invention

The present invention provides a method for manufacturing a skin-friendly pressure-sensitive adhesive composition. In a first aspect, the method comprises the steps of:
a. providing a matrix composition comprising a polymeric photoinitiator of the general formula I:

R₁(A₁)ᵣ-(R₂(A₂)ₘ-O)ₒ—(R₃(A₃)ₙ-O)ₚ—R₄(A₄)ₛ (I)
b. curing the matrix composition in step a. by exposing it to UV radiation.

As the photoinitiators are bound within the matrix composition after curing, the likelihood of photoinitiators of low molecular weight leaching from the surface of the cured material is reduced.

The matrix composition may additionally comprise one or more adhesive-forming polymers and/or adhesive-forming monomers. Alternatively, the matrix composition consists of the polymeric photoinitiator of the general formula I; i.e. the polymeric photoinitiator is the only component of the matrix composition.

In the polymeric photoinitiator of formula (I), R₂ and R₃ are independently at each occurrence identical or different, linear or branched alkylene or cycloalkylene groups; wherein R₂ and R₃ may be substituted with one or more substituents selected from CN; azides, esters; ethers; amides; halogen atoms; sulfones; sulfonic derivatives; NH₂ or Nalk₂, where alk is any C₁-C₈ straight chain alkyl group, C₃-C₈ branched or cyclic alkyl group. R₂ may be —CH₂CH₂—, in which one or more H atoms may be replaced by A₂. Similarly, R₃ may be —CH₂CH₂—, in which one or more H atoms may be replaced by A₃. As an alternative, R₂=—CH(CH₃)CH₂—, in which one or more H atoms may be replaced by A₂. R₃ may be —CH(CH₃)CH₂—, in which one or more H atoms may be replaced by A₃.

R₂ and R₃ can be selected from any alkylene group having up to 25 carbon atoms and include both branched and straight chain alkylene groups. Exemplary, non-limiting alkylene groups include methylene, ethylene, propylene, butylene, pentylene, hexylene, heptylene, octylene, nonylene, in the normal, secondary, iso and neo attachment isomers. Exemplary, non-limiting cycloalkylene groups include cyclopropylene, cyclobutylene, cyclopentylene and cyclohexylene.

As set out above, the alkylene groups R₂ and R₃ may be substituted with, apart from the photoinitiator moieties, substituents such as CN, azides, esters, ethers, amides, halogen atoms, sulfones, sulfonic derivatives, NH₂ or Nalk₂. “alk” is any C₁-C₈ straight chain alkyl group, C₃-C₈ branched or cyclic alkyl group. Photoinitiator moieties can be covalently linked to R₂ and/or R₃ as designated by R₂(A₂) and R₃(A₃), where A₂ and A₃ can be any of the photoinitiator moieties described herein.

R₁ and R₄ are independently at each occurrence identical or different, linear or branched alkyl or cycloalkyl groups or aryl groups or are independently at each occurrence selected from H, OH, CN, halogens, amines, amides, alcohols, ethers, thioethers, sulfones and derivatives thereof, sulfonic acid and derivatives thereof, sulfoxides and derivatives thereof, carbonates, isocyanates, nitrates, acrylates, polyethylenes, polyethylene oxides, polypropylene oxides, polyvinyl pyrrolidones, polypropylenes, polyesters, polyamides, polyacrylates, polystyrenes, and polyurethanes.

In some cases, when R₁ and R₄ are alkyl and aryl groups, they may be substituted with, apart from the photoinitiator moieties, substituents such as CN, OH, azides, esters, ethers, amides (e.g.—CONR′R″ or R′CONR″—, where R′ and R″ are alkyl groups, suitably C1-C25 alkyl groups), halogen atoms, sulfones, sulfonic derivatives, NH₂ or Nalk₂, where alk is any C1-C8 straight chain alkyl group, C3-C8 branched or cyclic alkyl group. Photoinitiator moieties can be covalently linked to R₁ and/or R₄ as designated by R₁(A₁) and R₄(A₄), where A₁ and A₄ can be any of the photoinitiator moieties described above.

R₁ and R₄ may independently be at each occurrence identical or different, linear or branched alkyl or cycloalkyl groups. R₁ and R₄ can be selected from any alkyl group having up to 25 carbon atoms and include both branched and straight chain alkyl groups. Exemplary, non-limiting alkyl groups include methyl, ethyl, propyl, butyl, pentyl, hexyl, heptyl, octyl, nonyl, in the normal, secondary, iso and neo attachment isomers. Exemplary, non-limiting cycloalkyl groups include cyclopropyl, cyclobutyl, cyclopentyl and cyclohexyl.

R₁ and R₄ can also be selected from aryl groups, such as any aromatic hydrocarbon with up to 20 carbon atoms. Exemplary, non-limiting aryl groups include phenyl, naphthyl, furanyl, thiophenyl, pyrrolyl, selenophenyl, and tellurophenyl. R₁ and R₄ can also be H, OH, CN, halogens, amines (e.g.—NR′R″, where R′ and R″ are alkyl groups, suitably C1-C25 alkyl groups), amides (e.g. —CONR′R″ or R′CONR″—, where R′ and R″ are alkyl groups, suitably C1-C25 alkyl groups), alcohols, ethers, thioethers, sulfones and derivatives thereof, sulfonic acid and derivatives thereof, sulfoxides and derivatives thereof, carbonates, isocyanates, nitrates, acrylates. R₁ is suitably OH. R₄ is suitably H.

Furthermore, R₁ and R₄ can be selected from polymeric entities. R₁ and R₄ may each independently be selected from the group consisting of polyacrylates, polyethylene oxides, polypropylene oxide, polyvinyl pyrrolidones, polyesters, polyamides and polyurethanes. The molecular weight of said polymeric entities is typically in the range of 50-50,000 Da.

The indices o and p are each a real number from 0-5000 provided that o+p>0. The indices o and p may each be from 0-3000, preferably 0-2000.

The indices m and n are each a real number from 0-10, provided that m+n>0, Suitably, m and n are each an integer of from 0-8, preferably 0-5, provided that m+n>0. More suitably, m=1 and/or n=1. Suitably, m+n≧1. In one aspect, m=1, n=0 and the ratio o:p is at least 1:1000, preferably at least 1:500.

The indices r and s are each a real number from 0-5. Suitably, r and s are each from 0-4, preferably 0-2. Suitably, r and s are independently 1 or greater, e.g. 1 or 2.

The indices m, n, o, p, r and s in the general formula I represent an average/sum and the formula I thereby represents alternating, periodic, statistical/random, block and grafted copolymers. The copolymer ABAAABABBABA having the formula A₇B₅ may be mentioned as an example of a random copolymer.

As an example of the identity of formula I applied to a polymeric photoinitiator described in the present invention is given in Scheme 1.

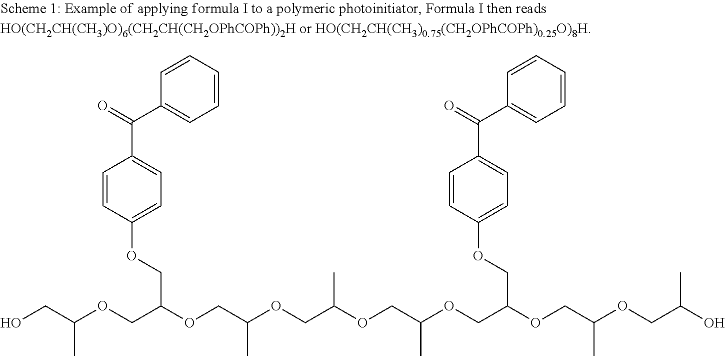 [SMILES: CC(O)COC(C)COC(COc1ccc(C(=O)c2ccccc2)cc1)COC(C)COC(C)COC(C)COC(COc1ccc(C(=O)c2ccccc2)cc1)COC(C)CO]

The polyalkylether photoinitiator according to the invention may have a molecular weight between 5 and 10,000 kDa, preferably between 10 kDa and 1,000 kDa, more preferably between 10 kDa and 500 kDa. In the present invention, M_{w} (the weight averaged molecular weight) is used to characterize the polymeric photoinitiators.

Efficiency of the polymeric photoinitiator is among other things related to how well the photoinitiator is blended with the adhesive-forming polymer(s) or monomer(s). Amongst important parameters in this respect is the molecular weight of the photoinitiator. A molecular weight which is too high does not allow for good miscibility of the polymeric photoinitiator with other components of the matrix composition. Important for the present invention is the miscibility of the polymeric photoinitiator with the other components in the matrix composition, when considering a two-component system. In particular, if the chemical nature and molecular weight of the polymeric photoinitiator and the adhesive-forming polymer are markedly different, a poor miscibility is obtained, which in turn results in a matrix composition that is difficult to cure.

### Photoinitiator Moieties

In Formula (I) above, A₁, A₂, A₃ and A₄ are identical or different photoinitiator moieties;

Photoinitiator moieties A₁, A₂, A₃ and A₄ may be linked to R₁, R₂, R₃, and R₄, respectively, via a spacer group. The spacer group may be selected from the group consisting of alkylene, cycloalkylene, aryl, and alkylene ether groups. The spacer group, if any, may be selected from the same functional groups as R′₁, R′₂, R′₃ and R′₄ and additionally from groups consisting of alkylethers, such as —(CH₂CH₂O)ₜ—, where t can be any integer of from 0-100.

In the present invention, a photoinitiator is defined as a moiety which, on absorption of light, generates reactive species (ions or radicals) and initiates one or several chemical reactions or transformation. One preferred property of the photoinitiator is good overlap between the UV light source spectrum and the photoinitiator absorption spectrum. Another desired property is a minor or no overlap between the photoinitiator absorption spectrum and the intrinsic combined absorption spectrum of the other components in the matrix. Good compatibility of the polymeric photoinitiator in the matrix consisting of material to be cured is also a property of interest.

In an embodiment of the polyalkylether photoinitiator according to the invention, A₁, A₂, A₃ and A₄ are identical or different photoinitiator moieties selected from the group consisting of benzoin ethers, phenyl hydroxyalkyl ketones, phenyl aminoalkyl ketones, benzophenones, thioxanthones, xanthones, acridones, anthraquinones, fluorenones, dibenzosuberones, benzils, benzil ketals, α-dialkoxy-acetophenones, α-hydroxy-alkyl-phenones, α-amino-alkyl-phenones, acyl-phosphine oxides, phenyl ketocoumarins, silane, maleimides, and derivatives thereof. The photoinitiator moieties A₁, A₂, A₃ and A₄ can also consist of derivatives of the photoinitiator moieties listed.

In an embodiment of the polyalkylether photoinitiator according to the invention, A₁, A₂, A₃ and A₄ are identical or different photoinitiator moieties selected from the group consisting of 2-hydroxy-2-methyl-propiophenone, benzophenone, thioxanthone, benzil, anthraquionone, camphorquinone, benzoin ether, acylphosphine oxide, silane, and derivatives thereof. The photoinitiator moieties A₁, A₂, A₃ and A₄ can also consist of derivatives of the photoinitiator moieties listed.

In an embodiment of the polyalkylether photoinitiator according to the invention, A₁, A₂, A₃ and A₄ are identical photoinitiator moieties. However, A₁, A₂, A₃ and A₄ may be at least two different photoinitiator moieties.

Suitably, at least one of A₁, A₂, A₃ and A₄ is a benzophenone photoinitiator moiety. At least A₂ and A₃ may be benzophenone photoinitiator moieties.

The photoinitiator moieties of the invention may independently be cleavable (Norrish Type I) or non-cleavable (Norrish Type II). Upon excitation, cleavable photoinitiator moieties spontaneously break down into two radicals, at least one of which is reactive enough to abstract a hydrogen atom from most substrates. Benzoin ethers (including benzil dialkyl ketals), phenyl hydroxyalkyl ketones and phenyl aminoalkyl ketones are important examples of cleavable photoinitiator moieties. The photoinitiator moieties of the invention are efficient in transforming light from the UV or visible light source to reactive radicals which can abstract hydrogen atoms and other labile atoms from polymers and hence effect covalent cross-linking. Optionally, amines, thiols and other electron donors can be either covalently linked to the polymeric photoinitiator or added separately or both. The addition of electron donors is not required but may enhance the overall efficiency of cleavable photoinitiators according to a mechanism similar to that described for the non-cleavable photoinitiators below.

Suitably, the photoinitiator moieties of the invention are all non-cleavable (Norrish Type II). Non-cleavable photoinitiator moieties do not break down upon excitation, thus providing fewer possibilities for the leaching of small molecules from the matrix composition. For reference, see e.g. A. Gilbert, J. Baggott: “Essentials of Molecular Photochemistry”, Blackwell, London, 1991). Excited non-cleavable photoinitiators abstract a hydrogen atom from an organic molecule or, more efficiently, abstract an electron from an electron donor (such as an amine or a thiol). The electron transfer produces a radical anion on the photoinitiator and a radical cation on the electron donor. This is followed by proton transfer from the radical cation to the radical anion to produce two uncharged radicals; of these, the radical on the electron donor is sufficiently reactive to abstract a hydrogen atom from most substrates. Benzophenones and related ketones such as thioxanthones, xanthones, anthraquinones, fluorenones, dibenzosuberones, benzils, and phenyl ketocoumarins are important examples of non-cleavable photoinitiators. Most amines with a C—H bond in α-position to the nitrogen atom and many thiols will work as electron donors. The photoinitiator moieties of the invention are preferably non-cleavable.

Self-initiating photoinitiator moieties are within the scope of the present invention. Upon UV or visible light excitation, such photoinitiators predominantly cleave by a Norrish type I mechanism and cross-link further without any conventional photoinitiator present, allowing thick layers to be cured. Recently, a new class of β-keto ester based photoinitiators has been introduced by M. L Gould, S, Narayan-Sarathy, T. E. Hammond, and R. B. Fechter from Ashland Specialty Chemical, USA (2005): “Novel Self-Initiating UV-Curable Resins: Generation Three”, Proceedings from RadTech Europe 05, Barcelona, Spain, Oct. 18-20 2005, vol. 1, p. 245-251, Vincentz. After base-catalyzed Michael addition of the ester to polyfunctional acrylates a network is formed with a number of quaternary carbon atoms, each with two neighbouring carbonyl groups.

Another self-initiating system based on maleimides has also been identified by C. K. Nguyen, W. Kuang, and C. A. Brady from Albemarle Corporation and Brady Associates LLC, both USA (2003): “Maleimide Reactive Oligomers”, Proceedings from RadTech Europe 03, Berlin, Germany, Nov. 3-5, 2003, vol. 1, p. 589-94, Vincentz. Maleimides initiate radical polymerization mainly by acting as non-cleavable photoinitiators and, at the same time, spontaneously polymerize by radical addition across the maleimide double bond. In addition, the strong UV absorption of the maleimide disappears in the polymer, i.e. maleimide is a photobleaching photoinitiator; this could make it possible to cure thick layers.

So, in an embodiment of the invention, the photoinitiator moieties include at least two different types of photoinitiator moieties. Preferably the absorbance peaks of the different photoinitiators are at different wavelengths, so the total amount of light absorbed by the system increases. The different photoinitiators may be all cleavable, all non-cleavable, or a mixture of cleavable and non-cleavable. A blend of several photoinitiator moieties may exhibit synergistic properties, as is e.g. described by J. P. Fouassier: “Excited-State Reactivity in Radical Polymerization Photoinitiators”, Ch. 1, pp. 1-61, in “Radiation curing in Polymer Science and technology”, Vol. II (“Photo-initiating Systems”), ed. by J. P. Fouassier and J. F. Rabek, Elsevier, London, 1993. Briefly, efficient energy transfer or electron transfer takes place from one photoinitiator moiety to the other in the pairs [4,4′-bis(dimethyl-amino)benzophenone+benzophenone], [benzophenone+2,4,6-trimethylbenzophenone], [thioxanthone+methylthiophenyl morpholinoalkyl ketone].

Furthermore, it has recently been found that covalently linked 2-hydroxy-1-(4-(2-hydroxyethoxy)phenyl)-2-methylpropan-1-one, which is commercially available with the trade name Irgacure 2959, and benzophenone in the molecule 4-(4-benzoylphenoxyethoxy)phenyl 2-hydroxy-2-propyl ketone gives considerably higher initiation efficiency of radical polymerization than a simple mixture of the two separate compounds, see S. Kopeinig and R. Liska from Vienna University of Technology, Austria (2005): “Further Covalently Bonded Photoinitiators”, Proceedings from RadTech Europe 05, Barcelona, Spain, Oct. 18-20 2005, vol. 2, p. 375-81, Vincentz. This shows that different photoinitiator moieties may show significant synergistic effects when they are present in the same oligomer or polymer.

Each and every one of the above-discussed types of photoinitiators and photoinitiator moieties may be utilised as photoinitiator moieties in the polymeric photoinitiators of the present invention.

### Polymeric Backbone (Photoinitiator Segment)

The polymeric backbone consists of a polyalkylether segment with the general formula —(R₂(A₂)ₘ-O)ₒ—(R₃(A₃)ₙ-O)ₚ— wherein R₂ and R₃ can be selected from any alkylene group having up to 25 carbon atoms and include both branched and straight chain alkylene and cycloalkylene groups. Exemplary, non-limiting alkylene groups include methylene, ethylene, propylene, butylene, pentylene, hexylene, heptylene, octylene, nonylene, in the normal, secondary, iso and neo attachment isomers. Exemplary, non-limiting cycloalkylene groups include cyclopropylene, cyclobutylene, cyclopentylene and cyclohexylene.

In an embodiment of the polyalkylether photoinitiator according to the invention R₂ and R₃ are independently —CH₂CH₂— in which one or more H atoms may be replaced by A₂ or A₃, respectively.

Inclusion of polypropylene oxide units in the polymer backbone improves the adhesive behaviour. In an embodiment of the polyalkylether photoinitiator according to the invention, therefore, R₂ and R₃ are independently —CH(CH₃)CH₂— in which one or more H atoms may be replaced by A₂ or A₃, respectively. Suitably, both R₂ and R₃ may be —CH(CH₃)CH₂—. In a particular embodiment, R₁ and R₄ may both be —CH(CH₃)CH₂—, in which one or more H atoms may be replaced by A₁ and A₄, respectively.

In some cases the alkylene groups may, apart from the photoinitiator moieties, bear substituents such as CN, azides, esters, ethers, amides (e.g.—CONR′R″ or R′CONR″—, where R′ and R″ are alkyl groups, suitably C1-C25 alkyl groups), halogen atoms, sulfones, sulfonic derivatives, NH₂ or Nalk₂, where alk is any C1-C8 straight chain alkyl group, C3-C8 branched or cyclic alkyl group. Photoinitiator moieties can be covalently linked to R₂ and/or R₃ as designated by R₂(A₂)ₘ and R₃(A₃)ₙ, where A₂ and A₃ can be any of the photoinitiator moieties described above. The indices m, n, o and p are as set out above.

### Polymeric Photoinitiators of the Invention

### Polyethylene Oxide Derived Photoinitiators.

The polymeric photoinitiators can be either synthesized by a polymerization reaction or photoinitiators can be grafted onto a polymeric backbone. A general scheme for a direct synthesis of a polymeric photoinitiator with pendant photoinitiator moieties based on epoxy-ring opening is shown in Scheme 2, where the symbols from the general formula for the polymeric photoinitiators are exemplified.

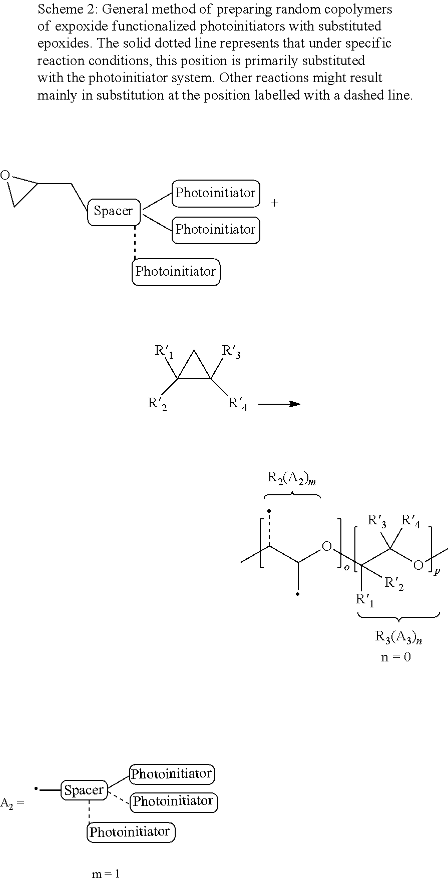

The epoxide functionality used for the polymerization is obtained through a reaction with epichlorhydrine, but might also be obtained through a reaction with an allyl-derivative which is then subsequently oxidized with an oxidizing agent such as m-chloro-perbenzoic acid or hydrogen peroxide.

As illustrated in Scheme 2, attack of a nucleophile, either the initiator or an alkoxide ion, occurs at the least substituted carbon atom on the epoxide present on the spacer group. Some reaction conditions, e.g. acidic conditions might favour the converse, meaning that the most substituted carbon atom on the epoxide is attacked by the nucleophile. For simplicity, only polymerizations resulting in attack of the least substituted carbon atom in the photoinitiator attached epoxide, is illustrated in the following, but the invention is not so limited.

With respect to substituents, R′₁, R′₂, R′₃ and R′₄ can be selected from any alkyl groups having up to 25 carbon atoms and includes both branched, cyclic and straight chain alkyl groups. Exemplary alkyl groups include methyl, ethyl, propyl, butyl, pentyl, hexyl, heptyl, octyl, nonyl, in the normal, secondary, iso and neo attachment isomers. R′₁, R′₂, R′₃ and R′₄ can also be selected from aryl groups, such as any aromatic hydrocarbon with up to 20 carbon atoms. Exemplary aryl groups include phenyl, naphthyl, furanyl, thiophenyl, pyrrolyl, selenophenyl, and tellurophenyl. In some cases the alkyl and aryl groups may bear substituents such as CN, azides, esters, ethers, amides (e.g.—CONR′R″ or R′CONR″—, where R′ and R″ are alkyl groups, suitably C1-C25 alkyl groups), halogen atoms, sulfones, sulfonic derivatives, NH₂ or Nalk₂, where alk is any C1-C8 straight chain alkyl group, C3-C8 branched or cyclic alkyl group. R′₁, R′₂, R′₃ and R′₄ may also be H.

As a first example, a polymerization of 2-hydroxy-2-methyl-1-(4-(2-(oxiran-2-ylmethoxy)ethoxy)phenyl)propan-1-one (3) with either itself or ethylene oxide results in a (co)-polymer which is a polymeric photoinitiator (Scheme 3). The precursor for the synthesis of this polymer is 2-hydroxy-1-(4-(2-hydroxyethoxy)phenyl)-2-methylpropan-1-one (Irgacure 2959). A synthesis of (3) is outlined in U.S. Pat. No. 5,744,512.

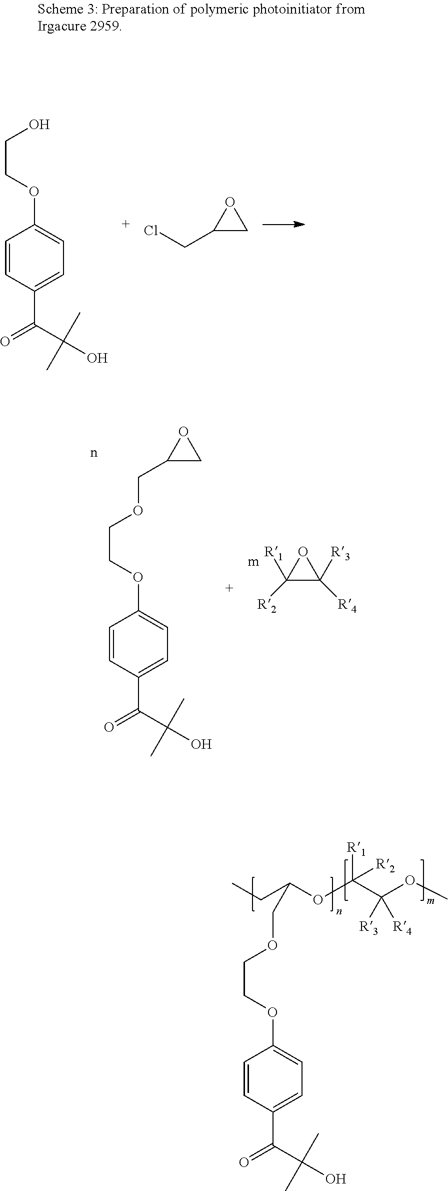

An alternate route to analogues of (3) is illustrated in Scheme 4, where the hydroxyalkyl phenone is formed in a Friedel-Crafts reaction with isobuturyl chloride as described in U.S. Pat. No. 5,744,512.

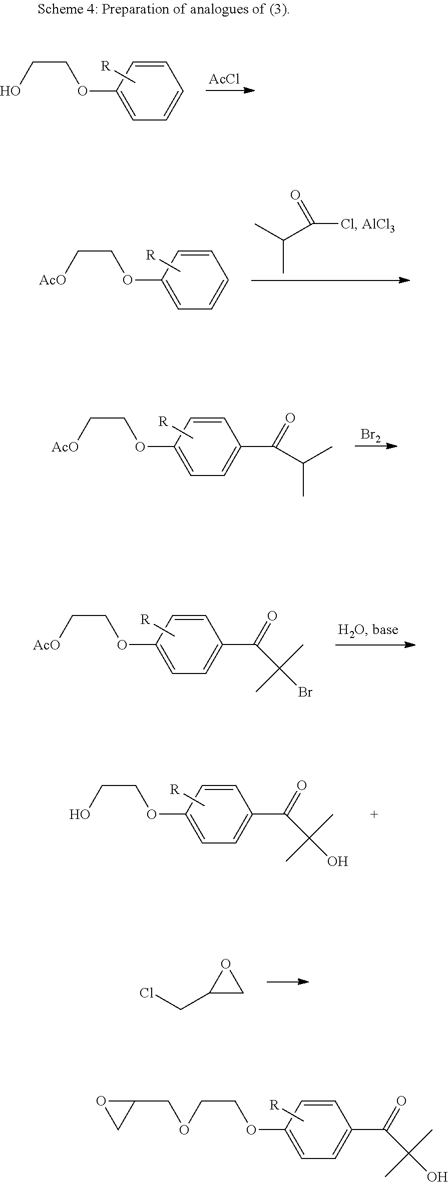

The synthesis of Irgacure 2959 has previously been described elsewhere (German Offenlegungsschrift 3.512.179). The attachment of photoinitiators with similar structure as Irgacure 2959 onto a polyalkylether is the main focus of the present invention. Following the synthetic route in Scheme 4, it will be possible to place specific substituents on the benzene ring by methods generally known in the art.

Derivatives of Irgacure 2959 are characterized as Type I photoinitiators, and other photoinitiators that falls in this category are benzoinethers, benzil ketals, α-dialkoxy-acetophenones, α-hydroxy-alkyl-phenones, α-amino-alkyl-phenones and acylphosphine oxides.

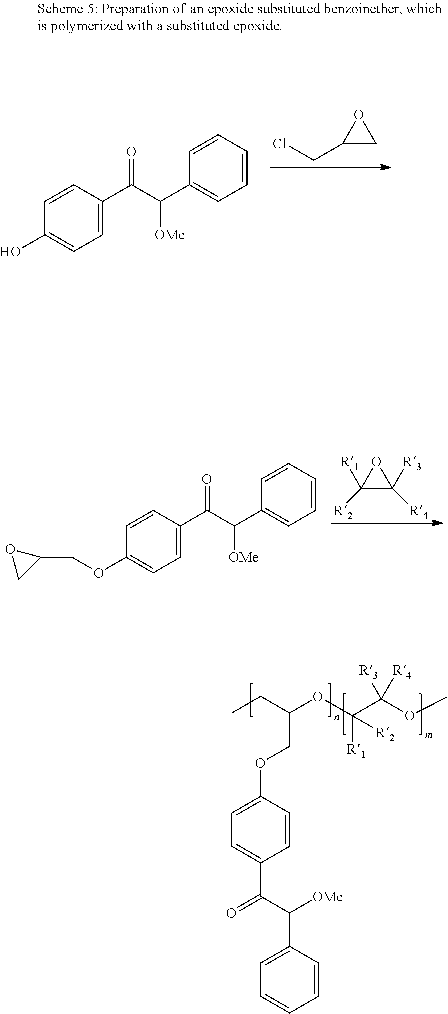

Depicted in Scheme 3-5 are examples of Type I photoinitiators attached to a polyalkylether backbone and an example of the preparation of a Type II polymeric photoinitiator is shown in Scheme 6 with xanthones, thioxanthones and acridones as the photoinitiator moiety itself.

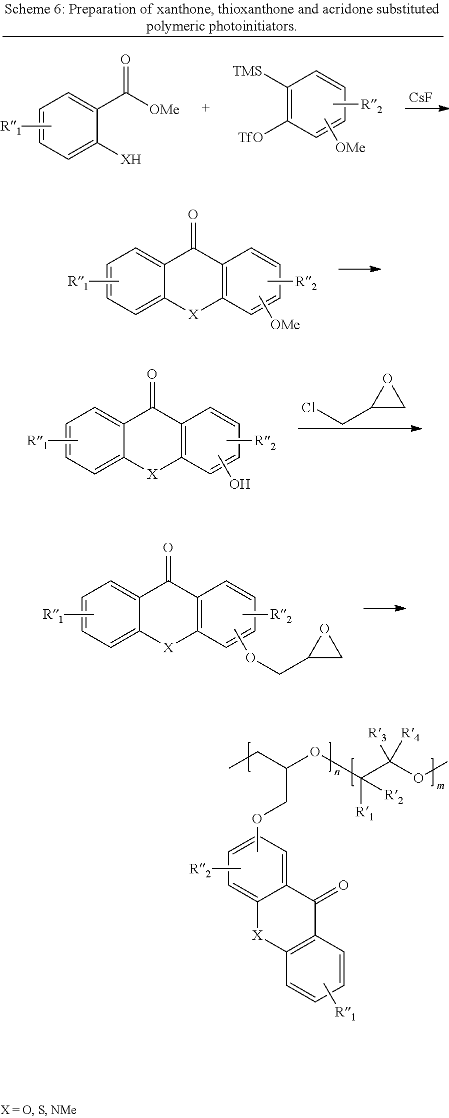

The preparation of the polymeric photoinitiator shown in Scheme 6, follows the same principles as shown in Scheme 3-5, where a hydroxy functionality present on the photoinitiator is reacted with epichlorhydrin. The resulting compound is then co-polymerized with a substituted epoxide thus resulting in the polymeric photoinitiator. Preparation of various substituted xanthone, thioxanthone and acridone molecules are detailed in J. Zhao, R. C. Larock J. Org. Chem. 72 (2007), 583-588. R″₁, and R″₂ may be selected from the same set of functional groups as R′₁, R′₂, R′₃ and R′₄.

As another example of a type II polymeric photoinitiator, a benzophenone substituted polyethylene oxide is illustrated in Scheme 7.

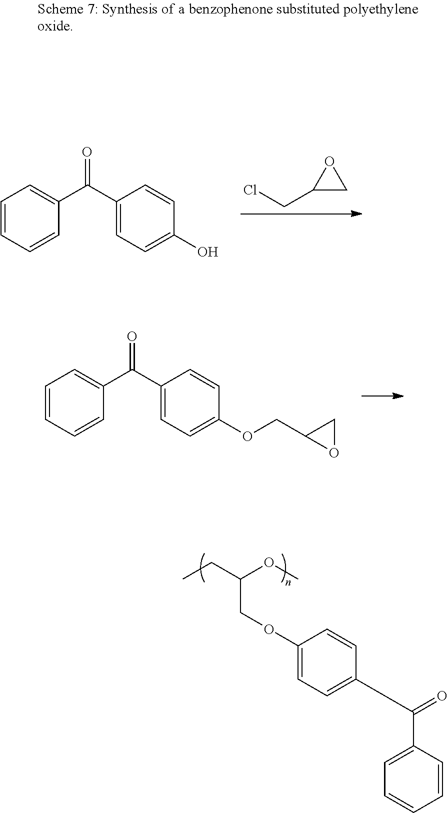

Synthesis of the epoxide derivatized benzophenone has been described for other analogues than the pure benzophenone in U.S. Pat. No. 4,376,788. No details were given for the intermediate in Scheme 7. The epoxide can subsequently be polymerized into the polyethylene oxide substituted benzophenone.

An alternate route to derivatized polypropylene oxide could be via grafting techniques as exemplified in Scheme 8.

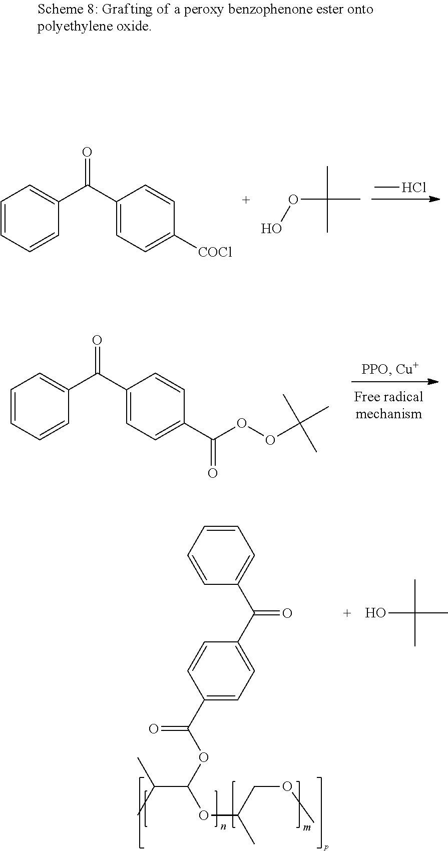

Grafting of the peroxy ester is catalysed by copper(I) as described in J. March: “Advanced Organic Chemistry. Reaction, Mechanisms, and Structure”, 3. ed., p. 636-7, Wiley-Interscience, New York, 1985.

### Polyalkyl Oxide Derived Photoinitiators

A general scheme for preparation of polyalkyl oxide derived photoinitiators is shown in Scheme 9, where the polymer is synthesized through an acyclic diene polymerization (ADMET) reaction.

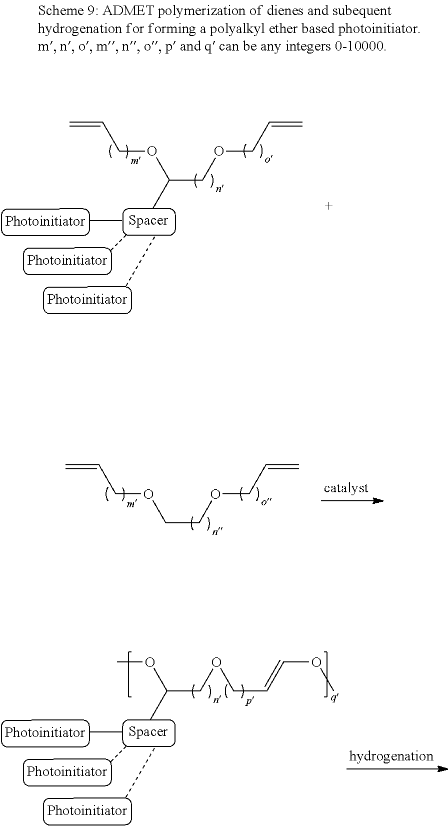

Such polymerization types have been described in K. B. Wagener, K. Brzezinska Macromolecules, 24 (1991), 5273-5277.

Considerable research has been focused on polymerizing substituted oxiranes, with different initiators and different solvents. Thus (4-(oxiran-2-ylmethoxy)phenyl)(phenyl)methanone can most likely be polymerized with e.g. potassium t-butoxide as an initiator in an anionic polymerization scheme as done in P. Yang, X. Zhu, Y. Yo, Y. M. Xia and T. Li Jour. Appl. Polym. Sci. 113 (2009), 3656-3660. Reaction conditions for similar polymerizations with a variety of other nucleophiles such as potassium hydroxide as initiators are presented in J. Cao, N.-F. Yang, P.-D. Wang and L.-W. Yang Polymer International, 57 (2008), 530-537. Several reaction conditions are published in patent literature as well, where in U.S. Pat. No. 4,472,560 metal cyanide complexes are used as catalysts for the epoxide polymerization. Organoaluminium catalysts are also described in U.S. Pat. No. 4,009,128 to work well in a cationic polymerization scheme.

### Matrix Composition

As set out above, the polymeric photoinitiators of formula (I) may be combined with one or more adhesive-forming polymers and/or adhesive-forming monomers in the matrix composition.

Adhesive-forming polymers are those which—upon curing with the polymeric photoinitiators of the invention—provide adhesive compositions. Curing creates cross-links between the adhesive-forming polymers and polymeric photoinitiators. In addition, cross-links may be formed internally between molecules of adhesive-forming polymers or polymeric photoinitiators. Suitably, the adhesive-forming polymer is selected from the group consisting of polyacrylates, polyalkylethers, polyurethanes, polyethylene vinyl acetates, polyvinylpyrrolidone and co-polymers and blends thereof.

Adhesive-forming monomers are monomers which—upon polymerization—provide adhesive-forming polymers, as described above. Suitable adhesive-forming monomers are selected from the group consisting of acrylate monomers, N-vinylpyrrolidone, and epoxide monomers.

For providing an adhesive after a curing step, a polymerization of the monomeric entities occurs in conjecture with cross-linking.

Other possible components in the matrix composition include anti-oxidants such as BHT (2,6-bis(1,1-dimethylethyl)-4-methylphenol), Irganox 1010 (from Ciba) and similar structures. Therapeutic additives are also possible components in the matrix composition. When such additional components are present in the matrix composition, they may be added directly at the same time as the matrix composition is formed, at any point prior to curing.

### Curing

Once the polymeric photoinitiator of the general formula I has been combined with one or more adhesive-forming polymers and/or adhesive-forming monomers to form a matrix composition, the matrix composition is cured by exposing it to UV radiation.

The ultraviolet spectrum is divided into A, B and C segments where UV A extends from 400 nm to 315 nm, UV B from 315 to 280 nm, and UV C from 280 to 100 nm. By using a light source that generates light with wavelengths in the visible region (400 to 800 nm) some advantages are obtained with respect to the depth of the curing, provided that the photoinitiator can successfully cure the material at these wavelengths. In particular, scattering phenomena are less pronounced at longer wavelength, thus giving a larger penetration depth in the material. Thus photoinitiators which absorb, and can induce curing, at longer wavelength are of interest. By judicially choosing substituents on the aromatic moieties, the absorption spectrum of the polymeric photoinitiator can to some extent be red-shifted, which would then facilitate curing at comparatively greater depths.

Multi-photon absorption can also be used to cure samples using light sources emitting at wavelengths twice or even multiple times the wavelength of light needed for curing in a one-photon process. For example, a composition containing a photoinitiator with an absorption maximum at ˜250 nm could possibly be cured with a light source emitting at ˜500 nm utilizing a two-photon absorption process provided that the two-absorption cross section is sufficiently high. A multi-photon initiated cure process could also facilitate greater spatial resolution with respect to the cured area, exemplified in Nature 412 (2001), 697 where a 3D structure is formed by a two-photon curing process.

In the present invention, curing is primarily initiated by exposing the matrix composition or polymeric photoinitiator to high energy irradiation, preferably UV light. The photoinitiated process takes place by methods described above and which are known per se, through irradiation with light or UV irradiation in the wavelength range from 250 to 500 nm. Irradiation sources which may be used are sunlight or artificial lamps or lasers. Mercury high-pressure, medium pressure or low-pressure lamps and xenon and tungsten lamps, for example, are advantageous. Similarly, excimer, solid stated and diode based lasers are advantageous. Even pulsed laser systems can be considered applicable for the present invention. Diode based light sources in general are advantageous for initiating the chemical reactions.

In the curing process, the polymeric photoinitiator transforms the matrix composition, in a chemical process induced by light.

### Auto-Curing

The polymeric photoinitiators described here can both facilitate curing of a surrounding matrix but since the photoinitiators themselves are polymers they can also “auto-cure”, meaning that the polymeric photoinitiators can solely constitute the matrix composition that is cured with UV irradiation. As such the pristine polymeric photoinitiator can be cured to form a cross-linked network, or the polymeric photoinitiator can be a constituent in a mixture which is subsequently cured to form a cross-linked network. This is particularly relevant when R₁ and R₄ are hydrophilic polymers such as e.g. polyacrylates, polyethylene oxides, polypropylene oxides, polyvinyl pyrrolidones, polyesters, polyamides and polyurethanes.

In one aspect, therefore, the invention provides a method for manufacturing a skin-friendly, pressure-sensitive adhesive in which the matrix composition consists of the polymeric photoinitiator of the general formula I, as defined in claim 1.

The “auto-curing” method described above suitably takes place with steps a. and b. occurring, directly after one another (i.e. with no intermediate steps). In one aspect of this “auto-curing” method, the method consists of steps a. and b. alone.

A one-component system—as provided by the “auto-curing” method—provides advantages, in that the polymeric photoinitiators are thermoplastic. As such, they become less viscous under higher shear rate, making them easier to process in an extrusion process. In contrast, for example, polyvinyl pyrrolidone cannot be extruded. All details and structural refinements of the polymeric photoinitiator provided herein are aimed at providing photoinitiators suitable for use in the “auto-curing” method.

In addition, the polymeric photoinitiators of the “auto-curing” method may comprise the sole component of the matrix composition; i.e. the matrix composition may consist of the polymeric photoinitiators. This provides the advantage that additives (e.g. plasticizers, viscosity modifiers) can be avoided, thereby reducing the chances of low molecular weight components from leaching from the cross-linked matrix composition.

### Adhesive Composition

Using the methods of the invention, a route to pressure-sensitive adhesives is achieved. Shear resistance, tack and peel strength can be used to characterize pressure sensitive adhesives, all which may be measured with a rheometer. Shear resistance and peel strength relate to the material's long-time flow behaviour, whereas tack is a measure of the ability to spontaneously form a bond to another material under light pressure within a short application time. In particular with respect to tack, a low-lying tan δ peak and a low value of G′ supplemented by a low amount of cross-links at 1 Hz (a high tan δ value) results in high tack. The requirements for achieving a high shear resistance are high G′ values and high viscosities at lower frequencies (<0.1 Hz). High peel strengths can be achieved by having high G″ values at higher frequencies (>100 Hz).

Additional components may be added to the composition such as tackifier resin, plasticisers and wax. However, as the polymeric photoinitiators themselves are adhesive, the matrix composition may simply consist of the polymeric photoinitiators of the invention. In other words, no additional components are added, which further reduces the risk of leaching of substances from the adhesive.

In one embodiment of the invention, the adhesive composition further comprises a tackifying resin such as natural, modified or synthetic resins preferably polar resins such as rosins, rosin esters, hydrogenated rosins, hydrogenated rosin esters, and derivatives of such polar resins or pure aromatic monomer resins.

Tackifying resins can be added to control tack in the adhesives, i.e. reduce G′ and G″, and increase glass transition temperature.

The content of the tackifying resin is 0-40% (w/w) of the final adhesive. Preferably, the adhesive is substantially free of resin. When the adhesive composition contains resin, the content of the tackifying resin is preferably 0.1-40% (w/w) of the final adhesive and more preferably 10-20% (w/w) of the final adhesive.

In one embodiment of the present invention, the adhesive composition comprising polar plasticising oils and resin in the content of above 50% (w/w) of the final adhesive.

In one embodiment of the invention, the adhesive composition further comprises an additional plasticiser selected from the group of mineral oil, citrate oil, paraffin oil, phatalic acid esters, adepic acid esters (e.g. DOA), and liquid or solid resin.

In another embodiment of the invention, the adhesive composition further comprises a polyethylene wax.

Other ingredients may be added for auxiliary benefits. This could be antioxidants and stabilisers, fillers for rheology modification or active components like vitamin E or ibuprofen.

In another embodiment of the invention, the adhesive composition further comprises other ingredients selected from the group of antioxidants, stabilisers, fillers, pigments, flow modifiers, and active ingredients.

The adhesive composition according to the invention is tolerant for beta sterilisation, which means that it does not significantly degrade or change properties during beta sterilisation at a reasonable level.

The invention also relates to a skin-friendly pressure-sensitive adhesive composition obtainable via the methods described herein. The adhesives of the invention may be used for fixation applications, e.g. as adhesives for medical tapes, band aids and fixation of pads, foams or needles, providing good adhesion, high breathability and sterilisation tolerance.

### Medical Device

One aspect of the invention provides a medical device comprising the adhesive compositions of the invention. In particular, the medical device suitably comprises the adhesive composition of the invention and a backing layer.

The term “medical device” should be interpreted in a fairly broad sense. The medical device comprising an adhesive composition according to the invention may be an ostomy appliance, a dressing (including wound dressings), a wound drainage bandage, a skin protective bandage, a device for collecting urine (e.g. uridome), an orthose or a prosthese, e.g. a breast prothesis, and a faecal management device.

The medical device may also be a tape (e.g an elastic tape or film), or a dressing or a bandage, for securing a medical device, or a part of the medical device to the skin, or for sealing around a medical device attached to the skin.

The medical device may in its simplest construction be an adhesive construction comprising a layer of the pressure sensitive adhesive composition according to the invention and a backing layer.

The backing layer is suitably elastic (has a low modulus), enabling the adhesive construction to conform to the skin movement and provide comfort when using it.

In a preferred embodiment of the invention, the backing material has a structured surface to improve the adhesion between the adhesive and the backing material. Particularly preferred are backing materials where the molted adhesive can penetrate and create mechanical interlocking with for example Non Woven and non-woven film laminates.

The thickness of the backing layer used according to the invention is dependent on the type of backing used. For polymer films, such as polyurethane films, the overall thickness may be between 10 to 100 μm, preferably between 10 to 50 μm, most preferred about 30 μm.

According to a further embodiment, the invention relates to a medical device such as a thin adhesive dressing, wherein the thickness of the adhesive layer is between 50 and 250 μm where it is thickest. The adhesive layer may thus have varying thickens or it may have a uniform thickness selected from values between 50 and 250 μm.

A dressing of the invention may in a preferred embodiment comprise an absorbing pad for the uptake of body fluids, especially wound exudates, so as to enable the wound dressing to keep a constant moist environment over the wound site and at the same time avoid maceration of the skin surrounding the wound.

A dressing of the invention is optionally covered in part or fully by one or more release liners, or cover films to be removed before or during application. A protective cover or release liner may for instance be siliconised paper. It does not need to have the same contour as the dressing and a number of dressings may be attached to a larger sheet of protective cover. The release liner may be of any material known to be useful as a release liner for medical devices.

The protective cover is not present during the use of the dressing of the invention and is therefore not an essential part of the invention. Furthermore, the dressing of the invention may comprise one or more “non touch” grip(s) known per se for applying the dressing to the skin without touching the adhesive layer. Such a non-touch grip is not present after application of the dressing. For larger dressings it is suitable to have 2 or 3 or even 4 “non-touch” grips.

In another aspect, the invention relates to a wafer for an ostomy appliance comprising an adhesive construction as described above.

An ostomy appliance of the invention may be in the form of a wafer forming part of a two-piece appliance or in the form of a one-piece appliance comprises a collecting bag for collecting the material emerging from the stoma. A separate collecting bag may be attached to the wafer by any manner known per se, e.g. through mechanical coupling using a coupling ring or through use of adhesive flanges.

A wafer for an ostomy appliance of the invention also typically comprises a release liner as discussed above.

An ostomy appliance of the invention may be produced in a manner known per se from materials conventionally used for the preparation of ostomy appliances.

In a further embodiment, the invention relates to prosthesis of the type to be adhered to the skin of the user, such as a breast prosthesis comprising an adhesive construction according to the invention.

The invention also relates to a urine collecting device comprising an adhesive construction as described above.

Urine collecting devices according to the invention may be in the form of uri-sheaths.

In another embodiment of the invention, the adhesive is part of a faecal-collecting device, attaching a bag or another collecting device to the perianal skin.

The medical device may be coated on at least a surface portion thereof with the adhesive composition described herein. In some embodiments, the adhesive composition covers the full (outer) surface of the medical device, and in some other embodiments, only to a part of the surface thereof. In the most relevant embodiments, the adhesive composition covers at least a part of the surface (preferably the whole surface) of the medical device that—upon proper use—comes into direct contact with body parts for which the medical device is intended to be adhered.

The skilled person will be aware of suitable amounts, location and chemical makeup of the adhesive composition which will provide the desired skin-friendly effects.

### EXAMPLES

### Example 1

4-hydroxybenzophenone (Sigma-Aldrich) is reacted with 2-chloromethyl-2-methyl-oxirane (for example from O&W Pharmlab, LLC) in a 1:1 stoichiometry resulting in the formation of (4-((2-methyloxiran-2-yl)methoxy)phenyl)(phenyl)methanone. A mixture of this oxiran and 2-methyloxirane is prepared and polymerized under acidic conditions at 80° C. leaving a copolymer of 2-methyloxirane and (4-((2-methyloxiran-2-yl)methoxy)phenyl)(phenyl)methanone as a solid.

### Example 2

An oblate of pristine poly-co-2-methyloxirane-(4-((2-methyloxiran-2-yl)methoxy)phenyl)(phenyl)methanone is placed between the two plates in a rheometer (parallel plate configuration, bottom plate is a quartz glass plate). The distance between the plates is set to 0.3 mm and the temperature to 120° C. The measurements are run with fixed strain of 1% and a constant frequency of 1 Hz. When the loss and storage modules stabilize, a UV-lamp is turned on, thus irradiating the sample through the bottom plate on the rheometer via a fibre from the lamp. The loss and storage modules are then followed as a function of time, while the UV-lamp irradiated the sample. The evolvement of the storage and loss modulus displayed a significant change as a function of UV exposure.

1. A method for manufacturing a skin-friendly pressure-sensitive adhesive composition, said method comprising the steps of:
a. providing a matrix composition comprising a polymeric photoinitiator of the general formula I:
R₁(A₁)ᵣ-(R₂(A₂)ₘ-O)ₒ—(R₃(A₃)ₙ-O)ₚ—R₄(A₄)ₛ (I)
wherein R₂ and R₃ are independently at each occurrence identical or different, linear or branched alkylene or cycloalkylene groups; wherein R₂ and R₃ may be substituted with one or more substituents selected from CN; azides, esters; ethers; amides; halogen atoms; sulfones; sulfonic derivatives; NH₂ or Nalk₂, where alk is any C₁-C₈ straight chain alkyl group, C₃-C₈ branched or cyclic alkyl group; R₁ and R₄ are independently at each occurrence identical or different, linear or branched alkyl or cycloalkyl groups or aryl groups or are independently at each occurrence selected from H, OH, CN, halogens, amines, amides, alcohols, ethers, thioethers, sulfones and derivatives thereof, sulfonic acid and derivatives thereof, sulfoxides and derivatives thereof, carbonates, isocyanates, nitrates, acrylates, polyethylenes, polyethylene oxides, polypropylene oxides, polyvinyl pyrrolidones, polypropylenes, polyesters, polyamides, polyacrylates, polystyrenes, and polyurethanes; and when R₁ and R₄ are alkyl and aryl groups, they may be substituted with one or more substituents selected from CN; OH; azides; esters; ethers; amides; halogen atoms; sulfones; sulfonic derivatives; NH₂ or Nalk₂, where alk is any C₁-C₈ straight chain alkyl group, C₃-C₈ branched or cyclic alkyl group;
o and p are each a real number from 0-5000 provided that o+p>0;
m and n are each a real number from 0-10,
provided that m+n>0;
r and s are each a real number from 0-5; and
A₁, A₂, A₃ and A₄ are identical or different photoinitiator moieties; and
b. curing the matrix composition in step a. by exposing it to UV radiation.

2. The method according to claim 1, wherein the matrix composition additionally comprises one or more adhesive-forming polymers and/or adhesive-forming monomers.

3. The method according to claim 1, wherein the matrix composition consists of the polymeric photoinitiator of the general formula I, as defined in claim 1.

4. The method according to claim 1, wherein A₁, A₂, A₃ and A₄ are linked to R₁, R₂, R₃, and R₄, respectively, via a spacer group.

5. The method according to claim 4, wherein the spacer group is selected from the group consisting of alkylene, cycloalkylene, aryl, and alkylene ether groups.

6. The method according to claim 1, wherein R₂=—CH(CH₃)CH₂—, in which one or more H atoms may be replaced by A₂.

7. The method according to claim 1, wherein R₃=—CH(CH₃)CH₂—, in which one or more H atoms may be replaced by A₃.

8. The method according to claim 1, wherein R₁ and R₄=—CH(CH₃)CH₂—, in which one or more H atoms may be replaced by A₁ and A₄, respectively.

9. The method according to claim 1, wherein R₁═OH.

10. The method according to claim 1, wherein R₄═OH.

11. The method according to claim 1, wherein A₁, A₂, A₃ and A₄ are identical or different photoinitiator moieties selected from the group consisting of benzoin ethers, phenyl hydroxyalkyl ketones, phenyl aminoalkyl ketones, benzophenones, thioxanthones, xanthones, acridones, anthraquinones, fluorenones, dibenzosuberones, benzils, benzil ketals, α-dialkoxy-acetophenones, α-hydroxy-alkyl-phenones, α-amino-alkyl-phenones, acyl-phosphine oxides, phenyl ketocoumarins, silane, maleimides, and derivatives thereof.

12. The method according to claim 11, wherein A₁, A₂, A₃ and A₄ are identical or different photoinitiator moieties selected from the group consisting of 2-hydroxy-2-methyl-propiophenone, benzophenone, thioxanthone, benzil, anthraquionone, camphorquinone, benzoin ether, acylphosphine oxide, silane, and derivatives thereof.

13. The method according to claim 1, wherein A₁, A₂, A₃ and A₄ are identical photoinitiator moieties.

14. The method according to claim 1, wherein A₁, A₂, A₃ and A₄ are at least two different photoinitiator moieties.

15. The method according to claim 14, wherein at least one of A₁, A₂, A₃ and A₄ is a benzophenone photoinitiator moiety.

16. The method according to claim 15, wherein at least A₂ and A₃ are benzophenone photoinitiator moieties.

17. The method according to claim 1, wherein o and p are each from 0-3000, preferably 0-2000, provided that o+p>0.

18. The method according to claim 1, wherein m and n are each an integer from 0-8, preferably 0-5, provided that m+n>0.

19. The method according to claim 1, wherein m=1 and/or n=1.

20. The method according to claim 1, wherein m=1, n=0 and the ratio o:p is at least 1:1000, preferably at least 1:500.

21. The method according to claim 1, wherein r and s are each from 0-4, preferably 0-2.

22. The method according to claim 1, wherein the polymeric photoinitiator of formula (I) has a molecular weight between 5 kDa and 10,000 kDa, preferably between 10 kDa and 1,000 kDa, more preferably between 15 kDa and 500 kDa.

23. The method according to claim 1, wherein R₁ and R₄ are selected from the group consisting of polyacrylates, polyethylene oxides, polypropylene oxides, polyvinyl pyrrolidones, polyesters, polyamides and polyurethanes.

24. The method according to claim 1, wherein R₁ and R₄ are each independently selected from C₁-C₂₅ linear alkyl, C₃-C₂₅ branched alkyl and C₃-C₂₅ cycloalkyl.

25. The method according to claim 1, wherein the adhesive-forming polymer is selected from the group consisting of polyacrylates, polyalkylethers, polyurethanes, polyethylene vinyl acetates, polyvinylpyrrolidone and co-polymers and blends thereof.

26. The method according to claim 1, wherein the adhesive-forming monomer is selected from the group consisting of acrylate monomers, N-vinylpyrrolidone, and epoxide monomers.

27. The method according to claim 1, consisting of steps a. and b.

28. A skin-friendly pressure-sensitive adhesive composition obtainable via the method as defined in claim 1.

29. A medical device comprising the adhesive composition of claim 28.

30. The medical device according to claim 29, comprising a backing layer.
